(19) United States
(12) Patent Application Publication
(10) Pub. No.: US 2013/0245369 A1
(43) Pub. Date: Sep. 19, 2013

(54) LEFT ATRIAL APPENDAGE OCCLUSION SYSTEMS

(71) Applicant: SORIN CRM SAS, Clamart (FR)

(72) Inventor: Renzo Dal Molin, Chatillon (FR)

(73) Assignee: SORIN CRM SAS, Clamart (FR)

(21) Appl. No.: 13/831,939

(22) Filed: Mar. 15, 2013

Related U.S. Application Data
(60) Provisional application No. 61/611,380, filed on Mar. 15, 2012.

(51) Int. Cl. A61B 17/12

(52) U.S. Cl. CPC A61B 17/12109
USPC 600/104; 606/198

(57) ABSTRACT

A left atrial appendage (LAA) occlusion system and a method for occluding the LAA are disclosed. The LAA occlusion plug includes at least two expandable materials having shrunken and swollen states, two plates and a delivery tool configured to deliver the occlusion plug to the LAA. The LAA occlusion plug is configured to permanently occlude the LAA opening, such that blood clots cannot flow out of the LAA when the at least two hydrogels are in the swollen state. The LAA occlusion plug plates are configured to force the expandable materials to swell inwards in the axial direction and outwards in the radial direction such that the LAA occlusion plug is clipped to the LAA opening tissue.

### FIELD OF THE INVENTION

The invention relates generally to left atrial appendage (LAA) occlusion systems, and in particular to LAA occlusion systems and methods based on expandable materials.

### BACKGROUND OF THE INVENTION

During the last decade there has been a significant progress in the development of smart materials that target biological and bio-medical applications. Among those materials, polymer based hydrogels were investigated as they can resemble normal physiological conditions due to their high water content. Water based gels may be used for controlled drug delivery and release systems for example.

In addition, hydrogels are widely used in tissue engineering. Apart from the above mentioned bio-medical applications, hydrogels may be used as precise biosensors and diagnostic devices.

Polyelectrolyte hydrogels, which are polymer hydrogels with charged groups incorporated into their macromolecular network, are capable of volumetric and/or mechanical changes in the presence of external stimuli like electric field, pH or specific salt solution due to their chemical structure.

In case of electro-activated hydrogels, control over swelling, shrinking and bending behavior in response to external fields may be used to achieve direct conversion of electrical energy into mechanical energy. This makes polyelectrolyte hydrogels good candidates for biomedical applications.

Hydrogels may be used for the treatment of cardiovascular diseases requiring vessel occlusion, device sealing or cavity filling, taking advantage of the hydrogels ability to change form and shape due to the presence of external stimuli like electric field or environmental parameter changes like temperature and pH.

The LAA is a muscular pouch connected to the left atrium of the heart. In atrial fibrillation patients, blood clots arise from the left atrial appendage in more than 90% of the patients. The blood clots may dislodge forming emboli which may lead to damage to the brain, kidneys, or other organs supplied by the circulation system.

It would be highly advantageous to develop LAA occlusion systems that may be used to occlude the LAA opening preventing strokes in atrial fibrillation patients based on expandable materials.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1 illustrates a hydrogel based occlusion delivery system including a hemostasis valve, introducer sheath and dilator, according to certain embodiments;

FIGS. 2A-2D illustrate the guide insertion, dilator extraction and the occlusion protrusion set insertion over a guidewire, according to certain embodiments;

FIGS. 3A-3B illustrate a LAA occlusion plug, according to certain embodiments;

FIG. 4 illustrates a hydrogel protrusion set that includes electro-activating electrodes, according to certain embodiments;

FIG. 5 illustrates hydrogel based LAA occlusion method in a flowchart, according to certain embodiments; and

FIG. 6 illustrates the electro-activated hydrogel (EAH) based LAA occlusion method in a flowchart, according to certain embodiments.

### SUMMARY OF THE INVENTION

A LAA occlusion system and a method for occluding a LAA are disclosed. The LAA occlusion plug includes at least two expandable materials having shrunken and swollen states, two plates and a delivery tool configured to deliver the occlusion plug to the LAA. The LAA occlusion plug is configured to permanently occlude the LAA opening, such that blood clots cannot flow out of the LAA when the at least two expandable materials are in the swollen state. The LAA occlusion plug plates are configured to force the expandable materials to swell inwards in the axial direction and outwards in the radial direction such that the LAA occlusion plug is clipped to the LAA opening tissue.

In one embodiment, the expandable materials are selected from the group consisting of: hydrogels, electro-activated hydrogels, electro-activated polymers, carbon nano tubes based materials, piezoelectric materials and any other artificial muscle materials.

In another embodiment, the delivery tool comprises an endoscope miniaturized camera and a blood removal arrangement used for measuring the diameter of the LAA opening and for validating the fixation of the LAA occlusion plug to the LAA opening at the expandable materials swollen state.

In one embodiment, the first of the at least two expandable materials may be molded with a bulge and a second of the at least two expandable materials may be molded with a cavity such that when the expandable materials swell to their equilibrium state the bulge and the cavity mate, thereby strengthening the LAA occlusion plug fixation to the LAA opening tissue.

In another embodiment, the expandable materials may be molded in a substantially U shape profile, and wherein the at least two expandable materials may be rotated by 90 degrees to each other, such that in their swollen state the two rotated U shape expandable materials lock on each other, thereby strengthening the LAA occlusion plug fixation to the LAA opening tissue.

In one embodiment, the expandable materials have a central hole along their cylindrical axis, wherein the delivery tool is configured to deliver the LAA occlusion plug to the LAA over a guide wire configured to thread through the central hole.

In one independent embodiment, a LAA occlusion method is disclosed. The method includes providing a LAA occlusion plug with at least two expandable materials with shrunken and swollen states, two plates and a delivery tool; delivering the occlusion plug to the LAA in order to block blood clots to flow out of the LAA, wherein the at least two expandable materials are in their shrunken state; detaching the LAA plug from the delivery tool after the at least two expandable materials expanded to their swollen state and occlude permanently the LAA. The plates are configured to force the expandable materials to swell inwards in the axial direction and outwards in the radial direction clipping the LAA plug to the LAA opening tissue; and removing the delivery tool from the patient body.

In one embodiment, the method comprising further measuring the diameter of the LAA opening with an endoscope miniaturized camera and selecting the diameter of the expandable materials at their swollen state accordingly.

In another embodiment, the method comprising further validating the fixation of the LAA occlusion plug to the LAA opening tissue using the endoscope miniaturized camera before detaching the LAA occlusion plug from the delivery tool.

In one embodiment, the method comprising further removing the blood from the LAA using a blood removal arrangement allowing clear view of the LAA opening using the endoscope miniaturized camera.

In another embodiment, the expandable materials are selected from the group consisting of: hydrogels, electro-activated hydrogels, electro-activated polymers, carbon nano tubes based material, piezoelectric materials and any other artificial muscle materials.

In one embodiment, the LAA occlusion plug expandable materials are electro-activated materials and wherein the delivery tool comprising at least two electrodes, and wherein the method further comprising stimulating the at least two electro-activated expandable materials to their shrunken state.

Additional features and advantages of the invention will become apparent from the following drawings and description.

### DESCRIPTION OF THE PREFERRED EMBODIMENTS

A LAA occlusion system and a method for occluding a LAA are disclosed. The LAA occlusion plug may include at least two expandable materials having shrunken and swollen states, two plates and a delivery tool configured to deliver the occlusion plug to the LAA. The LAA occlusion plug is configured to permanently occlude the LAA opening, such that blood clots cannot flow out of the LAA when the at least two hydrogels are in the swollen state. The LAA occlusion plug plates are configured to force the expandable materials to swell inwards in the axial direction and outwards in the radial direction such that the LAA occlusion plug is clipped to the LAA opening tissue. As used in the present disclosure, the term “clip” means that the LAA opening tissue is positioned in between the at least two expandable materials. The LAA occlusion plug thus maintains its position in relation to the LAA opening tissue since the clipped portion of the LAA opening tissue prevents the expandable materials from moving there against.

Optionally, the delivery tool comprises an endoscope miniaturized camera and a blood removal arrangement used for measuring the diameter of the LAA opening and for validating the fixation of the LAA occlusion plug to the LAA opening tissue at the expandable materials swollen state.

The expandable materials may be hydrogels, electro-activated hydrogels, electro-activated polymers, carbon nano tubes based materials, piezoelectric materials and/or any artificial muscle materials known in the art.

Optionally, each expandable material is an electro-active hydrogel (EAH) and the LAA occlusion system, and/or the LAA occlusion delivery system, includes a stimulator for applying electrical stimulations onto the EAHs to transform them from state to state.

Optionally, the expandable materials are hydrogels wherein the hydrogel shrunken state is a substantially dried state and the swollen state is a wet state in equilibrium in fluid contact with blood.

Optionally, the hydrogel shrunken state is an electroactivatd contracted state and the hydrogel swollen state is an expanded state due to swelling with no electro-actuation applied and due to fluid contact with the patient blood.

### LAA Occlusion Systems

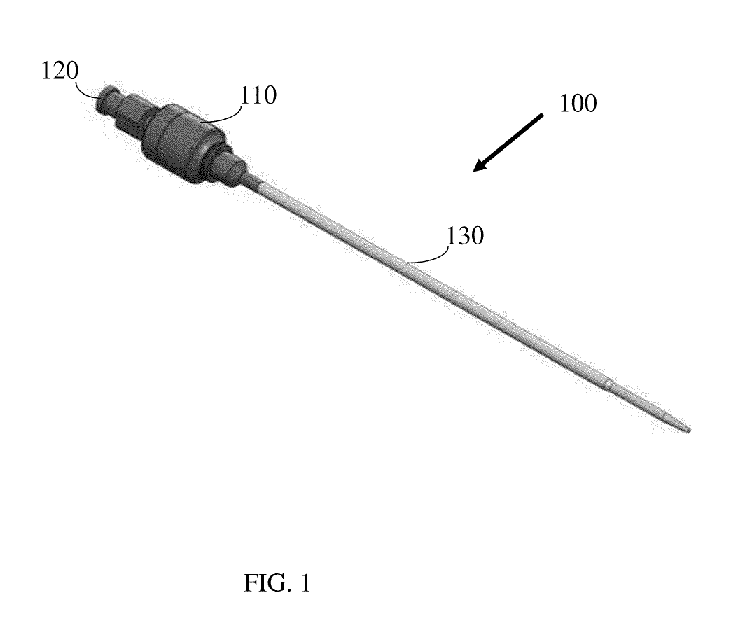

FIG. 1 illustrates a hydrogel based occlusion delivery system 100 that includes a hemostasis valve 110, introducer sheath 130 and dilator 120, according to certain embodiments. Occlusion delivery system 100 includes a basic pusher catheter and hydrogel protrusion set described further below. The hemostasis valve 110 has a wide entrance hole 120 for the insertion of dilator 120 in its proximal end. The introducer sheath 130 and dilator 120 are standard commercially available sheath and dilator typically 40-50 cm long and having a 7 French diameter. Other introducer sheath lengths and diameters may be used with embodiments of the present invention in order to match various sizes of LAAs to be occluded.

Optionally, the occlusion system may include an imaging means used to validate visually the positioning of an occlusion plug in the LAA opening wherein the delivery tool distal end may be made of an x-ray opaque material or the hydrogels may includes x-ray opaque material.

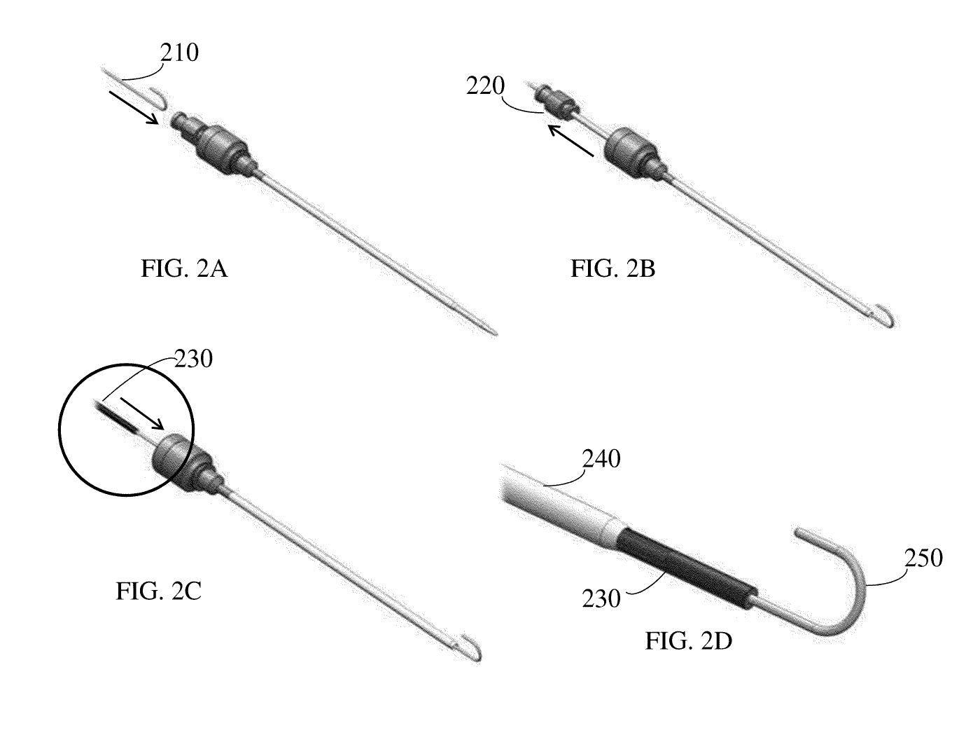

The step-by-step procedure for the delivery of the hydrogel based occlusion system is described in FIG. 2 below. The procedure starts with a needle insertion into a blood vessel, femoral or arterial approach may be used, followed by insertion of the introducer sheath 130 distal end to the blood vessel.

FIGS. 2A-2D illustrate the guidewire insertion, dilator extraction and the occlusion protrusion set insertion over a guidewire, according to certain embodiments. Guidewire 210 is inserted through a dilator 220 to the blood vessel until it reaches the LAA opening. Optionally, the guidewire has a J shape distal end 250 used to hold the LAA occlusion plug in the LAA opening before the expandable materials swell and occlude the LAA opening. After the guidewire in inserted, dilator 220 is extracted and an occlusion protrusion set 230 is inserted over the guidewire. The occlusion protrusion set 230 is installed over the proximal end of a guidewire 210 and pushed through the introducer sheath 250 while the guidewire 210 is held steady.

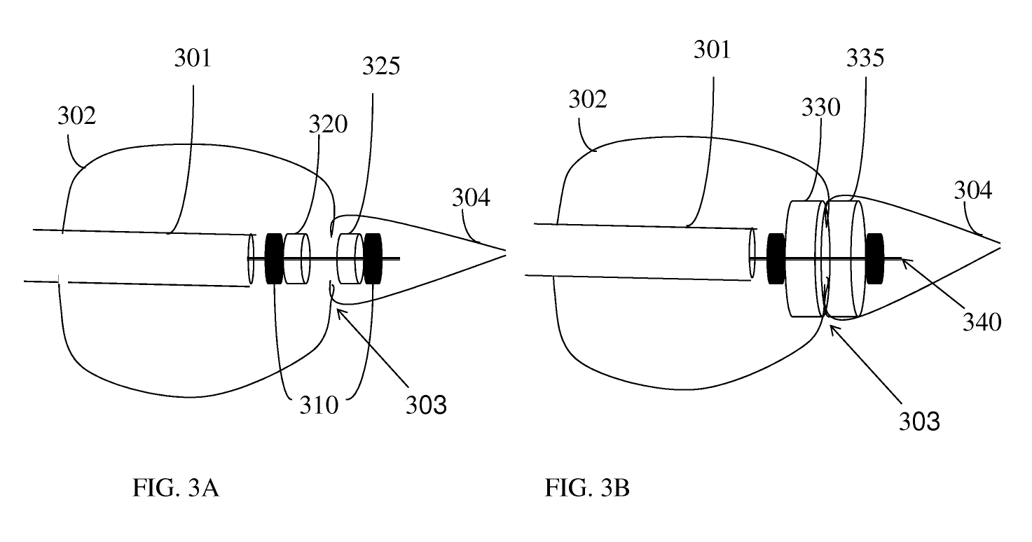

FIGS. 3A-3B illustrate a LAA occlusion plug, according to certain embodiments. The LAA occlusion plug, delivered through the delivery tool introducer sheath 301, may include two expandable materials 320 and 325 and two plates 310 and 315. The two expandable materials may be hydrogels for example, one hydrogel 325 is configured be positioned inside the LAA 304 and the second hydrogel 320 is configured to be positioned in the left atrium 302. The two expandable materials 320 and 325 are shown in their shrunken state before deployment. The two expandable materials are configured to swell inwards in the axial direction and outwards in the radial direction, as shown in 330, thereby occluding the LAA 304, as shown in FIG. 3B. The two expandable materials swelling is configured to clip the LAA plug to the LAA opening tissue 303. The two plates 310 and 315 force the hydrogels to swell inwards in the axial direction and outwards in the radial direction and to tighten the occlusion plug to the LAA opening tissue. Optionally, the two plates 310 and 315 may be further tightened with a screw (not shown) along the longitudinal axial axis 340. The two plates 310 and 315 may be metal plates or any other stiff material plates that may be implanted in a body. The diameter of the plates 310 and 315 is limited by the introducer sheath 301 diameter.

Optionally, expandable materials 320 and 325 each have a solid symmetrical shape around its longitudinal axis. Expandable materials 320 and 325 may have a substantially cylindrical shape and a central hole along their cylindrical axis enabling delivering the LAA plug over a guidewire configured to thread through the hydrogels central hole. In one embodiment, each of expandable materials 320 and 325 exhibits a 3 millimeters diameter in the shrunken state and 6 to 25 millimeter diameter in the swollen state.

The expandable materials may swell to their equilibrium swollen state 330 due to fluid contact with the patient blood and clip to the LAA opening tissue in their swollen state.

Optionally, the delivery tool includes an endoscope miniaturized camera and a blood removal arrangement used for measuring the diameter of the LAA opening and the diameter of the expandable materials, for example the hydrogels, may be selected accordingly.

Optionally, the endoscope miniaturized camera is used for validating the fixation of the LAA occlusion plug to the LAA opening tissue at the expandable materials swollen state before detaching the LAA plug from the delivery tool.

Optionally, the delivery tool includes a blood removal arrangement, such as saline injection, configured to clear the blood from the LAA allowing clear view of the LAA opening through the injected transparent saline using the endoscope miniaturized camera.

The occlusion LAA plug shown in FIGS. 3A-3B is a non limiting example of an expandable materials hydrogel based LAA occlusion plug configuration, based on two hydrogels that swell in fluid contact with blood and reach their equilibrium swollen state occluding the LAA.

The two hydrogels 320 and 325 may have the same diameters and widths or different diameters and widths.

Other configurations of expandable materials based LAA occlusion plug may be provided with different number and various shapes of expandable materials, without exceeding the scope. Optionally, the two expandable materials may be hydrogels that may be molded having bulges and matching cavities in the other hydrogel surface such that when the two hydrogels swell to their equilibrium swollen state, clipping the LAA opening tissue between the two hydrogel surfaces, the matching bulges and cavities close on each other strengthening the LAA occlusion plug fixation to the LAA opening tissue 303.

Optionally, two hydrogels 320 and 325 may be molded with a substantially U shape profile, where the two substantially U shape hydrogels are rotated by 90 degrees to each other in the LAA occlusion plug, such that in their swollen state the two rotated hydrogels locks on each other strengthening the LAA occlusion plug fixation to the LAA opening tissue.

The hydrogels shrunken state may be a substantially dried state and the hydrogels swollen state may be a swollen state in equilibrium in fluid contact with blood.

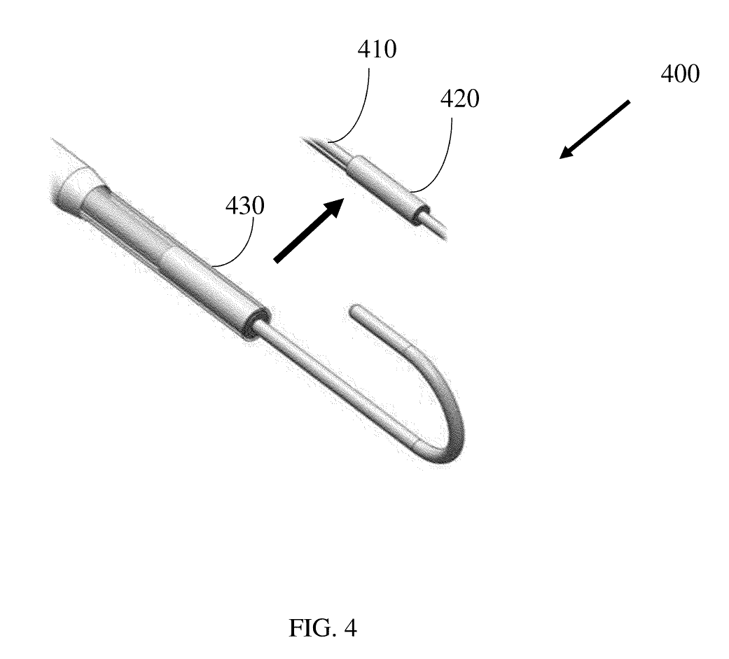

Optionally, the hydrogels shrunken state is an electro-activated contracted state and the hydrogels swollen state is an expanded state due to swelling with no electro-actuation applied and in equilibrium due to fluid contact with the patient blood as described further below regarding FIG. 4.

Optionally, the two expandable materials 320 and 325 may be EAHs and the delivery tool may include stimulating electrodes as shown in FIG. 4 below.

FIG. 4 illustrates a hydrogel protrusion set that includes electro-activating electrodes, according to certain embodiments. Hydrogel protrusion set 400 may include two or more electrodes used to electro-activate the hydrogel. The two or more electrodes may be a central electrode 410 mounted in the hydrogel central hole and an outer electrode 420 mounted on the catheter outer wall 430.

Optionally, more than one electrode may be mounted on the catheter outer wall 430. Optionally, the two or more electrodes may be mounted on the catheter wall 430 circulating the expandable materials without a central electrode.

Optionally, a stimulator unit (not shown) is provided with the LAA occlusion system. The electrical stimulation is arranged to maintain or change the state of expansion of the expandable materials.

Optionally, the expandable materials may be stimulated externally before deployment allowing applying high voltages (above 10 Volts) and high frequency AC signals (above 10 KHZ) onto the expandable materials.

According to embodiments of the present invention, the expandable material may be a Pluronic Methacrylic Acid Sodium Salt Hydrogel (PLMANa). PLMANa is biocompatible and non toxic and its basis polymer, Pluronic PF-127, is FDA approved. The PLMANa hydrogels are cross-linked, strengthened hydrogels designed to reach an equilibrium swollen state in blood environment. The PLMANa hydrogels are transparent and may be molded in various shapes in their shrunken state. The transparent PLMANa hydrogel may reach a diameter of about 6-25 mm in its swollen state in equilibrium in fluid contact with blood. Other shrunken state diameters and lengths and swollen state equilibrium diameters and lengths may be designed according to the required LAA opening sizes.

Optionally, LAA occlusion plug 300 may comprise other hydrogels, and other expandable materials without exceeding the scope.

### LAA Occlusion Methods

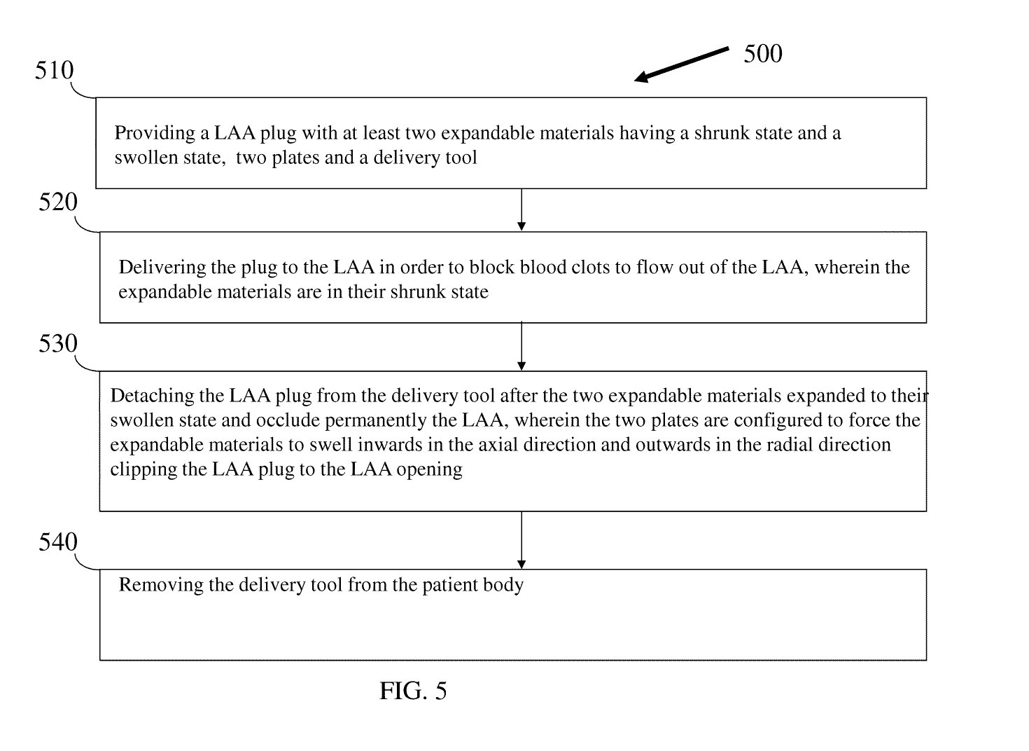

FIG. 5 illustrates the expandable materials based LAA occlusion method in a flowchart, according to certain embodiments. In stage 510, a LAA occlusion plug is provided with at least two expandable materials exhibiting shrunken and swollen states, two plates and a delivery tool; In stage 520, the LAA plug is delivered to the LAA wherein the at least two expandable materials are in their shrunken state; In stage 530, the LAA plug is detached from the delivery tool after the at least two expandable materials expanded to their swollen state, thereby occluding permanently the LAA blocking blood clots to flow out of the LAA.

The two plates are configured to force the expandable materials to swell inwards in the axial direction and outwards in the radial direction clipping the LAA plug to the LAA opening tissue; In stage 540, the delivery tool is removed from the patient body. Typically, the expandable materials, hydrogels for example, swell from their shrunken state to their equilibrium swollen state in the LAA in fluid contact with blood in 10-90 minutes.

Optionally, the LAA occlusion method 500 includes puncturing a central hole along the expandable materials cylindrical axis and delivering the LAA occlusion plug to the LAA over a guidewire. Optionally, the central hole along the cylindrical axis is performed by mechanical drilling of a substantially cylindrical shape expandable material in its dried shrunken state. Alternatively, a hydrogel may be molded with a central hole using appropriate molding cast having a central rod. Optionally, a molding cast with more than one central rod may be used in order to mold the hydrogel having more than one hole in order to increase swelling speed in the wet swollen state by increasing the hydrogel to fluid contact surfaces.

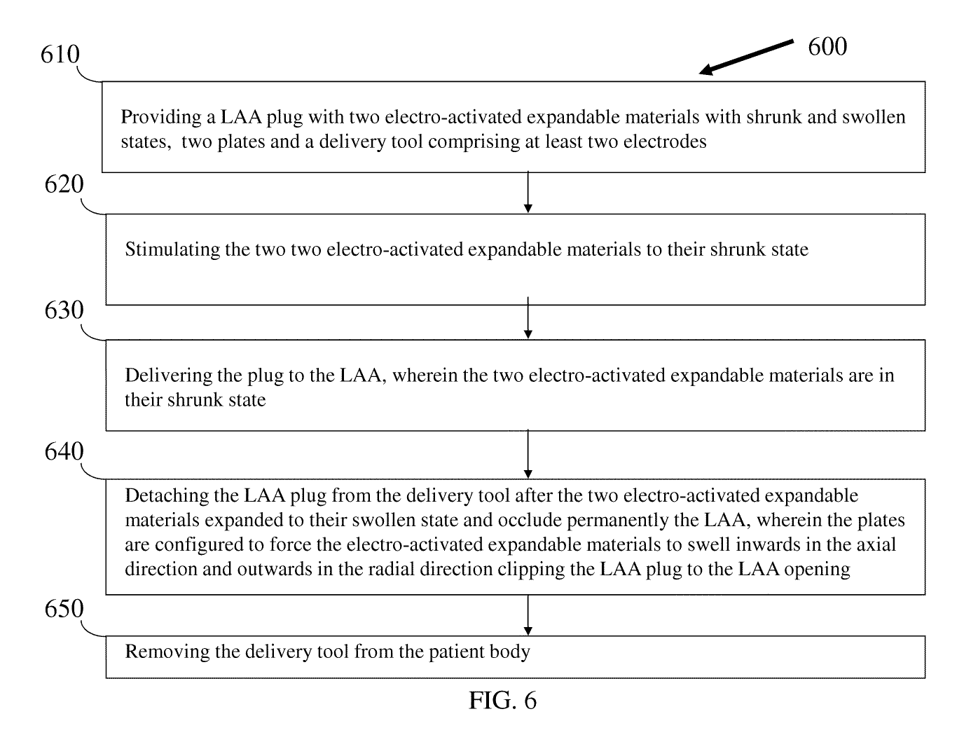

FIG. 6 illustrates an electro-activated expandable material based LAA occlusion method in a flowchart, according to certain embodiments. In stage 610, a LAA occlusion plug is provided with at least two expandable materials exhibiting shrunken and swollen states, two plates, a delivery tool that includes at least two electrode; In stage 620, the two expandable materials are stimulated to their shrunken state; In stage 630, the LAA plug is delivered to the LAA wherein the at least two expandable materials are in their shrunken state; In stage 640, the LAA plug is detached from the delivery tool after the at least two expandable materials expanded to their swollen state, thereby occluding permanently the LAA blocking blood clots to flow out of the LAA. The two plates are configured to force the expandable materials to swell inwards in the axial direction and outwards in the radial direction clipping the LAA plug to the LAA opening tissue; and In stage 650, the delivery tool is removed from the patient body.

Optionally, stimulating the electro-activated expandable material to its shrunken state 620 is performed before deployment outside of the patient body. Advantageously, stimulating the electro-activated expandable material before deployment allows applying high voltages and high frequency AC signals onto the electro-activated expandable material.

Alternatively, stimulating the electro-activated expandable material and keeping it in its shrunken state is performed during deployment of the electro-activated expandable material in the patient body through electrodes mounted in the delivery tool distal end.

Optionally, the LAA occlusion method may include measuring the diameter of the LAA opening with an endoscope miniaturized camera and selecting the diameter of the expandable materials at their swollen state accordingly.

Optionally, the LAA occlusion method may include validating the fixation of the LAA occlusion plug to the LAA opening tissue using the endoscope miniaturized camera before detaching the LAA occlusion plug from the delivery tool.

Optionally, the LAA occlusion method may include removing the blood from the LAA using a blood removal arrangement allowing clear view of the LAA opening using the endoscope miniaturized camera. Advantageously, the above described LAA occlusion system may be used to occlude the LAA in a minimal invasive procedure.

Another advantage of the LAA occlusion system described above is that the LAA opening may be measured and the fixation of the LAA occlusion plug to the LAA opening may be validated visually.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description. While preferred embodiments of the present invention have been shown and described, it should be understood that various alternatives, substitutions, and equivalents can be used, and the present invention should only be limited by the claims and equivalents thereof.

What is claimed is:

1. A left atrial appendage (LAA) occlusion system comprising:
a LAA occlusion plug comprising at least two expandable materials having shrunken and swollen states and two plates; and
a delivery tool configured to deliver said occlusion plug to the LAA,
wherein said LAA occlusion plug is configured to permanently occlude the LAA opening, such that blood clots cannot flow out of the LAA when said at least two expandable materials are in the swollen state, and
wherein said plates are configured to force the expandable materials to swell inwards in the axial direction and outwards in the radial direction such that the LAA occlusion plug is clipped to the LAA opening tissue.

2. The system according to claim 1, wherein said expandable materials are selected from the group consisting of: hydrogels, electro-activated hydrogels, electro-activated polymers, carbon nano tubes based materials, piezoelectric materials and any other artificial muscle materials.

3. The system according to claim 1, wherein said delivery tool comprises an endoscope miniaturized camera and a blood removal arrangement used for measuring the diameter of the LAA opening and for validating the fixation of said LAA occlusion plug to said LAA opening tissue at said expandable materials swollen state.

4. The system according to claim 1, wherein a first of said at least two expandable materials is molded with a bulge and a second of said at least two expandable materials is molded with a cavity such that when the expandable materials swell to their equilibrium state said bulge and said cavity mate, thereby strengthening the LAA occlusion plug fixation to the LAA opening tissue.

5. The system according to claim 2, wherein said expandable materials are molded in a substantially U shape profile, and wherein said at least two expandable materials are rotated by 90 degrees to each other, such that in their swollen state the two rotated U shape expandable materials lock on each other, thereby strengthening the LAA occlusion plug fixation to the LAA opening tissue.

6. The system according to claim 2, wherein said expandable materials have a central hole along their cylindrical axis, wherein said delivery tool is configured to deliver said LAA occlusion plug to the LAA over a guide wire configured to thread through said central hole.

7. A LAA occlusion method, the method comprising:
(a) providing a LAA occlusion plug with at least two expandable materials with shrunken and swollen states, two plates and a delivery tool;
(b) delivering said occlusion plug to the LAA in order to block blood clots to flow out of the LAA, wherein said at least two expandable materials are in their shrunken state;
(c) detaching said LAA plug from said delivery tool after said at least two expandable materials expanded to their swollen state and occlude permanently the LAA, wherein said plates are configured to force the expandable materials to swell inwards in the axial direction and outwards in the radial direction clipping the LAA plug to the LAA opening tissue; and
(d) removing the delivery tool from the patient body.

8. The method according to claim 7, the method comprising further measuring the diameter of the LAA opening with an endoscope miniaturized camera and selecting the diameter of said expandable materials at their swollen state accordingly.

9. The method according to claim 8, the method comprising further validating the fixation of said LAA occlusion plug to said LAA opening tissue using said endoscope miniaturized camera before detaching said LAA occlusion plug from said delivery tool.

10. The method according to claim 9, the method comprising further removing the blood from the LAA using a blood removal arrangement allowing clear view of the LAA opening using said endoscope miniaturized camera.

11. The method according to claim 7, wherein said expandable materials are selected from the group consisting of: hydrogels, electro-activated hydrogels, electro-activated polymers, carbon nano tubes based material, piezoelectric materials and any other artificial muscle materials.

12. The method according to claim 7, wherein said LAA occlusion plug expandable materials are electro-activated materials and wherein said delivery tool comprises at least two electrodes, and wherein the method further comprising stimulating said at least two electro-activated expandable materials to their shrunken state.
